(19) United States
(12) Patent Application Publication
(10) Pub. No.: US 2009/0258946 A1
(43) Pub. Date: Oct. 15, 2009

(54) ELECTROSTATICALLY CHARGED NASAL APPLICATION MULTIPURPOSE PRODUCTS AND METHOD

(75) Inventor: Ashok Wahi, Hillsborough, NJ (US)

Correspondence Address: STANLEY H. KREMEN, 4 LENAPE LANE, EAST BRUNSWICK, NJ, 08816, US

(73) Assignee: TRUTEK CORP., Hillsborough, NJ (US)

(21) Appl. No.: 12/489,185

(22) Filed: Jun. 22, 2009

Related U.S. Application Data
(60) Provisional application No. 61/078,472, filed on Jul. 7, 2008.

(51) Int. Cl. A61K 31/195; A61P 11/02

(52) U.S. Cl. 514/563

(57) ABSTRACT

A family of products designed to ameliorate sinusitis and allergic rhinitis, produced by combining certain chemical substances with existing products, thereby producing a synergistic effect and relief. The chemical substances create an electrostatically charged field around the nose and prevent allergens or pollutants from entering the nostrils to cause a reaction, illness, or discomfort for the user. Therefore, the Present Invention comprises products, which are applied to the face, and therefore around the nose and nasal passages, as well as those applied directly into the nose. These products include but are not limited to nasal sprays, rinses, washes, sunscreens, nasal strips, swabs (medicated and non-medicated), tissues, towelettes, cosmetics, and fragrances. Common nasal sprays often comprise saline solutions. Cosmetics comprise camouflage paint or theatrical makeup. The principle function of the Present Invention is to prevent harmful airborne particulates from entering the nose when either sprayed into the nose or applied to the face, nose, or nasal passages.

### CROSS REFERENCE TO RELATED APPLICATIONS

The Present Application is the non-provisional counterpart of U.S. Provisional Patent Application Ser. No. 61/078,472 (hereinafter the '472 Application) filed on Jul. 7, 2008. The Present Application claims the benefit of and priority to the 472′ Application which is incorporated by reference in its entirety herein.
The '472 Application discloses identical material to my prior U.S. Provisional Patent Application Ser. No. 60/598,462 (hereinafter the '462 Application) filed on Aug. 3, 2004 (now expired) entitled: “ELECTROSTATICALLY CHARGED NASAL APPLICATION MULTIPURPOSE PRODUCTS AND METHOD.” No new matter was added to the '462 Application to create the '472 Application. Said '462 Application is incorporated by reference in its entirety herein.
The Present Application is additionally related to my U.S. Pat. No. 5,468,488, entitled “ELECTROSTATICALLY CHARGED NASAL APPLICATION PRODUCT AND METHOD” issued on Nov. 21, 1995. This patent is incorporated by reference in its entirety herein.
The Present Application is further related to my U.S. Pat. No. 5,674,481, entitled “ELECTROSTATICALLY CHARGED NASAL APPLICATION PRODUCT” issued on Oct. 7, 1997. This patent is incorporated by reference in its entirety herein.
The Present Application is moreover related to my U.S. Pat. No. 6,844,005 entitled “ELECTROSTATICALLY CHARGED NASAL APPLICATION PRODUCT WITH INCREASED STRENGTH” issued on Jan. 18, 2005. This patent is incorporated by reference in its entirety herein.

### FIELD OF THE INVENTION

The Present Invention relates to products manufactured by combining chemical substances that create an electrostatic field capable of attracting or repelling airborne contaminants with products otherwise developed for various common uses in connection with the human body. One such product is a family of nasal sprays currently used for other purposes combined with cationic agents to enable restricting the flow of airborne contaminants into the nasal passages. This reduces the inflow of airborne contaminants to the nasal passages by capturing the contaminants and keeping them from entering the body. A second product is cosmetic makeup applied to the face, such as facial makeup, clown or theatrical makeup, or camouflage face paint. Such a product captures airborne contaminants, thereby preventing from entering the wearer's nasal passages.

### BACKGROUND OF THE INVENTION

In the Present Invention, chemical substances that create an electrostatic charged can be paired with products for different uses to create new products with properties that enhance the uses and effectiveness of the earlier products. Such products with desired electrostatic properties can be broadly categorized as:
Type I: Products that are created by imparting desired properties at an Atomic level. An example will be a new saline salt liquid of increased efficacy or strength.
Type II: Products that are created by imparting desired properties by mixing known chemical entities at a molecular level. For example, a common sunscreen product for the nose with increased charge to help from outdoor airborne particulate.
Type III: Applicators or means to apply pre-moistened or pre-dosed for single and/or multiple use by products under subject invention or for any of the above referenced prior arts. For example pre-moistened felt tip applicator in re-sealable foil pouch for one-day use.
Type IV: A hybrid of products of any two or more of the above Types. As examples—an anti-bacterial moisturizing gel soaked single use tissue with increased electrostatic properties to combat allergies or transmission of infectious particulates; or a camouflage face paint sponge applicator gel that will filter outdoor allergens from the air etc.

These multipurpose products can be formulated in the medium that is commonly associated with each of the paired products, or in any manner, such as cream, lotion, towelette or wipe, makeup or paint, spray, wash, gel, tissue, cotton swab, foam swab, mask, etc.

Prior art products were limited to capturing airborne particulates that were essentially responsible for hay fever and the like, as well as killing harmful microorganisms or preventing them from entering the nose. It did not combine the aforementioned use with other, unrelated, products applied to the face, or the nasal region.

Allergic rhinitis, the medical name for hay fever, is caused by body's reaction to tiny substances in the air called allergens. Allergens are generally harmless, and we breathe them in and out all the time. If a person suffers from allergies, the body believes these allergens are attacking the system. The body fights back by producing chemicals called histamines, which cause nasal passages to become irritated or inflamed. Allergic rhinitis generally does not create serious health problems, but it can make life very uncomfortable for those who have it.

Some of the most common symptoms of allergic rhinitis are congestion, itchy and/or runny nose, sneezing, and postnasal drip. These symptoms are body's natural response to the seasonal allergens or environmental irritants inhaled. Furthermore, rhinitis occurs during specific seasons, or continues throughout the year. How long the allergy symptoms continue may depend on type of rhinitis. Sinusitis often presents with similar symptoms.

Allergy and sinusitis sufferers may obtain relief using nasal sprays and they consist of the following five categories:
Nasal congestion
Allergies
Sinus infection
Runny noses
General stuffiness
   Furthermore, the sprays are subdivided into five main segments:
   Antihistamine Sprays
      Prescription only
      Most effective against dust and pollen allergies
   Corticosteroid Nasal Sprays
      Prescription only
      Targets congestion
      Inflammation
      Swelling
      Anti-inflammatory effect
   Chromolyn Sodium Nasal Sprays
      OTC
      Treats itchy eyes
      Runny noses
      Does not address stuffiness
   Decongestant Sprays
      OTC
      Helps reduce congestion
      Alleviates stuffiness
   Saline Nasal Spray/Saline Washes
      Safest of all nasal sprays
      Preventative measure for nasal problems
      Keep mucus from building up
      Keep the nose moist

Other products are applied to the face, such as nasal strips, fragrances, sunscreens, and cosmetics such as camouflage paint and facial makeup. These products have ordinary uses. They are applied to the faces of non-allergic persons as well as to those of allergy sufferers. If airborne particulate allergens can be prevented from entering the nose and contacting the nasal mucosa, allergic rhinitis could be greatly alleviated.

### SUMMARY OF THE INVENTION

The Present Invention is a group of new products designed to ameliorate symptoms of sinusitis and allergic rhinitis by combining certain chemical substances with existing products, thereby producing a synergistic effect. The chemical substances create an electrostatically charged field around the nose and prevent allergens or pollutants from entering the nostrils to cause a reaction, illness, or discomfort for the user. Therefore, the Present Invention comprises products, which are applied to the face, and therefore around the nose and nasal passages, as well as those applied directly into the nose. These products include but are not limited to nasal sprays, sunscreens, nasal strips, swabs (medicated and non-medicated), tissues, towelettes, cosmetics, and fragrances. Common nasal sprays often comprise saline solutions. Cosmetics comprise camouflage paint or facial makeup. These are but examples of the type of products with which these chemical substances may be effectively combined. The principle function of the Present Invention is to prevent harmful airborne particulates from entering the nose when either sprayed into the nose or applied to the face.

### DETAILED DESCRIPTION OF THE INVENTION

The multi-purpose products of the Present Invention possess the following properties:
Electrostatic charge to prevent allergens, pollutants, and other contaminants from entering the nasal passage, thereby greatly reducing allergic and other adverse reactions; and
Examples of products that are appropriate for application on the face and around the nostrils, include but are not limited to:
   NASAL SPRAYS. Nasal sprays are useful in providing relief for symptoms of allergies and colds, as well as for administering medication. By combining one of these nasal sprays with the electrostatic charge, a user will be able to either alleviate or treat the symptoms of allergies while at the same time preventing future allergic reactions.
   CAMOUFLAGE FACE PAINT. Camouflage face paint is used by troops while in combat in order to disguise them. The paint covers the entire face, and therefore is applied around the nose. This paint can be enhanced with an electrostatic charge in order to keep out dust and other allergens, preventing an allergy attack, which would be extremely useful for troops on duty.

ASTELIN® is the only prescription antihistamine spray approved to treat nasal symptoms triggered by both seasonal allergens and environmental irritants. Its active ingredient is Azelastine HCL. Inactive ingredients include:
Sodium Chloride,
Dibasic Sodium Phosphate,
Edelate Disodium,
Citric Acid,
Benzalkonium Chloride, and
Purified Water (pH 6.8).
   Nasonex® is an example of a Corticosteroid anti-inflammatory nasal spray used to treat the nasal symptoms of indoor and outdoor nasal allergies and year-round non-allergic nasal symptoms. Its active ingredient is Mometasone Furoate Monohydrate. Inactive ingredients include:
Glycerin,
Micro-Crystalline Cellulose,
Carboxy-Methylcellulose Sodium,
Sodium Citrate,
Citric Acid,
Benzalkonium Chloride, and
Polysorbate 80.
   Afrin® offers a variety of different products to treat nasal congestion. Their active ingredient is Oxymetazoline HCL (0.05%), a nasal decongestant. Inactive ingredients include:
Benzalkonium Chloride,
Benzyl Alcohol,
Edetate Disodium,
Polyethylene Glycol,
Povidone,
Propylene Glycol,
Sodium Phosphate Dibasic,
Sodium Phosphate Monobasic,
Camphor,
Eucalyptol,
Menthol,
Polysorbate 80
Chamomilla Recutita (Matricaria) Flower Oil (Flavor), and
Carboxy-Methylcellulose Sodium.
   NasalCrom® is an example of a Chromolyn Sodium nasal spray having Chromolyn Sodium as its active ingredient. Inactive ingredients include Benzalkonium Chloride, Edetate Disodium, and EDTA. Examples of saline solution nasal sprays are sold under the Xlear® brand name. Here, the active ingredients are saline and Xylitol. Inactive ingredients include Sodium Bicarbonate and grapefruit seed extract.

An example of camouflage paint would include:
Titanium Dioxide,
Mineral Oil,
Paraffin,
Beeswax,
Cornicia Cerifera Wax,
Micro Crystalline Wax,
Talc,
Propylparaben,
Corn Oil,
Propylene Glycol,
BHA,
BHT,
Propyl Gallate, and
Citric Acid.
   An alternate example of camouflage paint would include.
Ceresine Wax,
Castor Wax,
Mineral Oil (heavy viscosity),
Cosmetic Yellow,
Cosmetic Green (hydrous and anhydrous),
Cosmetic Black,
Cosmetic Burnt Sienna, and
Titanium Oxide (Atlas White).

Note that all of the nasal sprays listed above contain Benzalkonium Chloride, and neither of the camouflage paints contains that substance. For both products, Benzalkonium Chloride are either introduced or increased in amount to maximize the biocide effect, increase cationic skin adherence properties, and provide greater skin penetration to enhance the active ingredients. Similarly, Lysine HCL is introduced into the nasal sprays to act as a biocide and to act as a mucous membrane conditioner. Furthermore, special isotonic ingredients with moisturizing effects should be introduced. These include seed weed extracts and algae extracts.

Primarily, effective amounts of cationic agents are introduced to enhance the benefits of using the product. These cationic agents include without limitation:
Polyquaternium-6
Polyquaternium-7,
Polyquaternium-10,
Polyquaternium-22,
Polyquaternium-88
Cocodimonium Hydroxypropyl Hydrolyzed Keratin,
Hydroxypropyl Trimonium Hydrolyzed Soy Protein,
Hydroxypropyl Trimonium Silk Protein,
Hydroxypropyl Trimonium Wheat Protein, and
Hydroxypropyl Trimonium Oat Protein.

A number of effective nasal sprays have been developed by the Inventor to clear blocked sinuses. One such spray comprised 1% by weight of Polyquaternium-10 in aqueous solution as the active ingredient. Another comprised 0.75% by weight of Polyquaternium-6 in aqueous solution, which was found to be more effective.

The existing products often, but not always, comprise preservatives and other biocidic substances. The final products should contain these materials. Examples are anti-microbal and biopharg agents. Typical of these are Benzalkonium Chloride and Lysine HCL, respectively.

In addition, the aforementioned chemical substances may be introduced to wipes, towelettes, and wet tissues, all to be used for application to the face.

In summary, products such as saline solutions, fragrances, sunscreens, moisturizers, cosmetics, camouflage paint, and surgical masks, in the form of a cream, lotion, gel, liquid, spray, mist, wash, swab, strip, wipe, towelette, tissue, stick, or any other method can be integrated appropriately with electrostatic charge adequate for the intended uses.

I claim:

1. A manufactured product that is topically applied in a person's nostrils or to the person's face proximate to the nasal region,
said product comprising various chemical substances,
wherein said product is created by modifying an initial product, and
wherein the improvement to the initial product comprises incorporation of an effective amount of additional chemical substances that create an electrostatic field in the person's nostrils or proximate to the person's nasal region
such that similarly charged airborne particulates are repelled from, and oppositely charged airborne particulates are attracted to the person's nostrils or nasal region
whereby the person's symptoms of sinusitis or allergic rhinitis are ameliorated.

2. The product of claim 1, wherein said product is selected from the group consisting of:
nasal sprays,
nasal rinses,
nasal washes,
fragrances for application to the person's face,
makeup cosmetics for application to the person's face,
wet towelettes,
wet tissues, and
stick applicators.

3. The product of claim 2 wherein said product is camouflage face paint or theatrical makeup.

4. The product of claim 1, wherein the physical state of the product is selected from the group consisting of:
a liquid,
a cream,
a lotion,
a gel, and
an aerosol.

5. The product of claim 1 wherein at least one of the additional chemical substances is a cationic agent.

6. The product of claim 5 wherein the cationic agent is selected from the group consisting of:
Polyquaternium-6,
Polyquaternium-7,
Polyquaternium-10,
Polyquaternium-22,
Polyquaternium-88,
Cocodimonium Hydroxypropyl Hydrolyzed Keratin,
Hydroxypropyl Trimonium Hydrolyzed Soy Protein,
Hydroxypropyl Trimonium Silk Protein,
Hydroxypropyl Trimonium Wheat Protein, and
Hydroxypropyl Trimonium Oat Protein.

7. The product of claim 1 wherein at least one of the additional chemical substances is an anti-microbial agent.

8. The product of claim 7 wherein the anti-microbial agent is Benzalkonium Chloride.

9. The product of claim 1 wherein at least one of the additional chemical substances is a biopharg.

10. The product of claim 9 wherein the biopharg is Lysine HCL.

11. A nasal spray solution comprising a cationic agent, such that when the solution is sprayed into a person's nostrils, an electrostatic field is created in the person's nostrils and proximate thereto, whereby the effect of harmful airborne particulates to the person is ameliorated.

12. The nasal spray solution of claim 11 wherein the cationic agent is selected from the group consisting of:
Polyquaternium-6,
Polyquaternium-7,
Polyquaternium-10,
Polyquaternium-22,
Polyquaternium-88,
Cocodimonium Hydroxypropyl Hydrolyzed Keratin,
Hydroxypropyl Trimonium Hydrolyzed Soy Protein,
Hydroxypropyl Trimonium Silk Protein,
Hydroxypropyl Trimonium Wheat Protein, and
Hydroxypropyl Trimonium Oat Protein.

13. The nasal spray solution of claim 11 further comprising an anti-microbial agent.

14. The nasal spray solution of claim 13 wherein the anti-microbial agent is Benzalkonium Chloride.

15. The nasal spray solution of claim 11 further comprising a biopharg.

16. The nasal spray solution of claim 15 wherein the biopharg is Lysine HCL.
